(19) United States
(12) Patent Application Publication
(10) Pub. No.: US 2026/0365621 A1
(43) Pub. Date: Nov. 19, 2026

(54) Non-Hallucinogenic Tryptamine Compounds

(71) Applicant: SINTALICA S.R.L., Arezzo (IT)

(72) Inventors: Anna Notti, Messina (IT); Luigi Mondello, Messina (IT); Luca Santi, Rome (IT); Illaria Cacciotti, Rome (IT); Salvatore Cuzzocrea, Messina (IT)

(21) Appl. No.: 19/119,741

(22) PCT Filed: Dec. 22, 2023
(86) PCT No.: PCT/IB2023/063185

§ 371 Date: Apr. 9, 2025

(30) Foreign Application Priority Data
Dec. 23, 2022 (IT) 102022000026703

(51) Int. Cl. C07D 209/16; A61K 31/01; A61K 31/015; A61K 31/047; A61K 31/122; A61K 31/4045; A61K 31/437; A61K 31/4748; A61P 25/04

(52) U.S. Cl. CPC C07D 209/16; A61K 31/01; A61K 31/015; A61K 31/047; A61K 31/122; A61K 31/4045; A61K 31/437; A61K 31/4748; A61P 25/04

(57) ABSTRACT

The present invention relates to novel non-hallucinogenic tryptamine compounds of formula I and/or formula II:

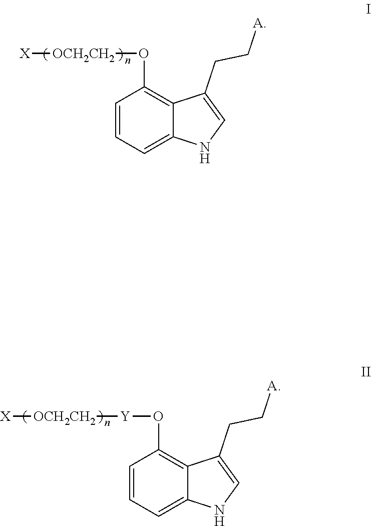

Compounds of formula I or II can be obtained by a process comprising a conjugation step of a non-hydrolysable chemical moiety with tryptamine precursors, which can be obtained synthetically, and which are analogue to tryptamines naturally occurring in psilocybin containing mushrooms, preferably in psilocybin containing mushrooms belonging to the Psilocybe genus. The conjugated non-hydrolysable chemical moiety allows to reduce or to prevent the crossing of the blood brain barrier (BBB).

### FIELD OF INVENTION

The present invention relates to non-hallucinogenic tryptamine compounds, preparation, pharmaceutical compositions and uses thereof.

### BACKGROUND OF THE INVENTION

Psilocybin, a tryptamine alkaloid isolated from various genera of fungi including the genus Psilocybe, and its active metabolite Psilocin, are known to bind to the 5-hydroxy tryptamine (serotonin) receptor 5-HT_{2A} in the Central Nervous System (CNS) causing hallucinogenic, anxiolytic, and psychoactive activities. Although psilocybin and its derivatives are currently under investigation among others for the treatment of major depression, anxiety, alcoholism and nicotine addiction, the psychedelic activity of these molecules represents a serious side effect for the development of new therapeutic agents. Recently, it has been proposed that psychedelics could be helpful in the treatment of neuroinflammation and chronic pain providing relief where conventional analgesics cannot (“The psychedelic remedy for chronic pain”, Clare Watson, Nature (2022) vol. 609, S100-S102 (DOI: 10.1038/d41586-022-02878-3); “From psychiatry to neurology: Psychedelics as prospective therapeutics for neurodegenerative disorders”, Urszula Kozlowska, Charles Nichols, Kalina Wiatr and Maciej Figiel, Journal of Neurochemistry (2022), vol. 162, p. 89-108 (DOI: 10.1111/jnc.15509).

Apart from CNS, high levels of 5-HT_{2A} receptor expression in other body areas such as the intestine, platelets and endothelial cells suggest that 5-HT_{2A} receptor play crucial roles in other aspects of physiology. This makes peripheral 5-HT_{2A} receptors promising targets for the development of new molecules with powerful anti-inflammatory properties.

Recent findings demonstrated that activation of 5-HT_{2A} receptors determined an anti-inflammatory response in rat model of asthma (“Structure-Activity Relationship Analysis of Psychedelics in a Rat Model of Asthma Reveals the Anti-Inflammatory Pharmacophore”, Thomas W. Flanagan, Gerald B. Billac, Alexus N. Landry, Melaine N. Sebastian, Stephania A. Cormier and Charles D. Nichols ACS Pharmacol. Transl. Sci. (2021), vol. 4, p. 488-502 (DOI: 10.1021/acsptsci.0c00063) and tumor necrosis factor alpha (TNF-α) mediated inflammation (Serotonin 5-HT_{2A} Receptor Activation Blocks TNF-α Mediated Inflammation In Vivo, Felix Nau Jr, Bangning Yu, David Martin, Charles D. Nichols, PLOS ONE (2013), vol. 8 (10): e75426 https://journals.plos.org/plosone/article?id=10.1371/journal.pone.0075426). In addition, it has been found that activation of 5-HT_{2A} receptors reduces neuropathic pain after spinal cord injury (Activation of 5-HT_{2A} Receptors Restores KCC2 Function and Reduces Neuropathic Pain after Spinal Cord Injury, Irene Sánchez-Brualla, Pascale Boulenguez, Cécile Brocard, Sylvie Liabeuf, Annelise Viallat-Lieutaud, Xavier Navarro, Esther Udina, Frédéric Brocard, Neuroscience (2018), vol. 387, p. 48-57, https://www.sciencedirect.com/science/article/pii/S0306452217305997).

Some attempt have been done in order to reducing the rate of transport of small molecules across the BBB (WO2005058367, describing the covalent attachment of a water-soluble polymer to small molecule drugs; WO2003032990, disclosing polymer conjugates of opioid antagonists with a polymer, such as poly(ethylene glycol).

In this context, tryptamine derivatives which are unable to cross the BBB in order to modulate the activity of peripheral 5-HT_{2A} receptors are needed, representing ideal molecules for the development of non-hallucinogenic anti-inflammatory drugs.

### DEFINITIONS

Unless otherwise defined, all the terms of the art, notations and other scientific terms used herein are intended to have the meanings commonly understood by those who are experts in the technique to which this description belongs. In some cases, terms with commonly understood meanings are defined here for clarity and/or for ready reference; the inclusion of these definitions in this description should therefore not be interpreted as representing a substantial difference with respect to what is generally understood in the art.

The terms “comprise”, “have”, “include”, “contain”, “comprising”, “having”, “including” and “containing” are to be understood as open terms (i.e. the meaning “comprising, but not limited to”) and are to be considered as a support also for terms such as “essentially consist of”, “consisting essentially of”, “consist of” or “consisting of”.

For all the ranges indicated in the text and in the claims of the present patent application, it is understood that the extremes of these ranges are included.

The tryptamine precursors used as starting points in the present invention can be obtained synthetically and are analogue to the tryptamines naturally occurring in psilocybin containing mushrooms, preferably in psychedelic psilocybin containing mushrooms belonging to the Psilocybe genus.

The psychedelic psilocybin containing mushrooms include a polyphyletic, informal group of fungi that contain psilocybin, psilocin or both within their biomass, typically within their fruiting bodies, resulting in their activation of a psychedelic reaction in a subject.

Preferred tryptamines naturally occurring in psilocybin containing mushrooms which can be used in the present are non-phosphorylated tryptamines, like psilocin, norpsilocin, 4-hydroxytriptamine and/or 4-hydroxy-N, N,N-trimethyltryptamine, and combinations thereof.

The pharmaceutically acceptable salts, derivatives, hydrate, or solvate of the above cited tryptamines naturally occurring in psilocybin containing mushrooms are comprised in the definition too.

The term “Psilocybe genus” may refer to the following non-limiting examples of suitable mushrooms containing psilocybin-like psychedelic compounds: Psilocybe atlantis, Psilocybe azurenscens, Psilocybe bohemica, Psylocibe baeocystis, Psilocybe cyanescens, Psilocybe cubensis, Psilocybe tampanensis, Psilocybe hoogshagenii Psilocybe mexicana, Psilocybe ovoideocystidiata, Psilocybe semilanceata Psilocybe weraroa, Psilocybe stuntzii, Psilocybe cyanofibrillosa, Psilocybe zapotacorum, Psilocybe yungensis, Psilocybe liniformans, Psilocybe xalapensis, Psilocybe venenata, Psilocybe subtropicalis, Psilocybe singer, Psilocybe schultesii, Psilocybe rostrata, Psilocybe quebecensis, Psilocybe pintonii, Psilocybe puberula, Psilocybe mairei, Psilocybe laurae, Psilocybe kumaenorum, Psilocy beheimii, Psilocy begalindoi, Psilocybe fmetaria, Psilocy beegonii, Psilocybe dumontii, Psilocybe carbonaria, Psilocybe cordispora, Psilocybe bispora, Psilocybe aucklandii, and combinations thereof.

The term “pharmaceutically acceptable salts or derivatives” herein refers to those salts or derivatives which possess the biological effectiveness and properties of the salified or derivatized compound and which and which do not produce adverse reactions when administered to a mammal, preferably a human. The pharmaceutically acceptable salts may be inorganic or organic salts; examples of pharmaceutically acceptable salts include but are not limited to: carbonate, hydrochloride, hydrobromide, sulphate, hydrogen sulphate, citrate, maleate, fumarate, trifluoroacetate, 2-naphthalenesulphonate, and para-toluenesulphonate. Further information on pharmaceutically acceptable salts can be found in Handbook of pharmaceutical salts, P. Stahl, C. Wermuth, WILEY-VCH, 127-133, (2008), herein incorporated by reference. The pharmaceutically acceptable derivatives include the esters, the ethers and the N-oxides.

“Psilocybin” is the common name of 4-phosphoryloxy-N,N-dimethyltryptamine.

“Psilocin” is the common name of 4-hydroxy-N,N-dimethyltryptamine.

“Norpsilocin” is the common name of 4-hydroxy-N-methyltryptamine.

The term “MAOIs” means monoamine oxidases inhibitors. The MAOIs used in the present invention belong to the β-carboline class of inhibitors and are selected from norharmane, perlolyrine, harmol, cordysinins tetrahydroharmine, 6-methoxyharmalan, harmalan, harmaline, harmalol, dihydro-β-carbolines (DHβC), tetrahydro-β-carboline (THβC), methyl-tetrahydro-β-carboline MTHβC, pinoline, 1-trichloromethyl-1,2,3,4-tetrahydro-b-carboline (TaClo), 6-methoxytetrahydroharmalan, ethyl β-carboline-3-carboxylate (βCCE), β-carboline-3-carboxylate (βCCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, lucartamides D, plakortamines C, eudistomidins, threctandramine, fascaplysin and/or salts, derivatives, hydrate, or solvate and/or combinations thereof.

The “carotenoids” also called “tetraterpenoids”, are yellow, orange, and red organic pigments that are produced by plants and algae, as well as several bacteria, and fungi. Carotenoids can be further categorized into two classes, xanthophylls (which contain oxygen) and carotenes (which are purely hydrocarbons and contain no oxygen). Examples of carotenes are α-carotene, β-carotene, lycopene, astaxantin and zeaxanthin. All carotenoids are derivatives of tetraterpenes, meaning that they are produced from 8 isoprene molecules and contain 40 carbon atoms. In general, carotenoids absorb wavelengths ranging from 400 to 550 nanometers (violet to green light).

Carotenoids that contain unsubstituted beta-ionone rings (including β-carotene, α-carotene, β-cryptoxanthin, and γ-carotene) have vitamin A activity (meaning that they can be converted to retinol).

Preferably, the carotenoids used in the present invention are obtained by plants extraction and/or microbial fermentation.

### SUMMARY OF INVENTION

The present invention relates to novel non-hallucinogenic tryptamine compounds of formula I and/or formula II:

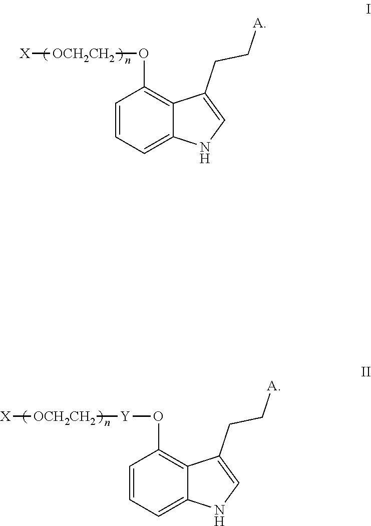

The invention relates also to a process for the preparation of compounds of formula I or II comprising a conjugation step of a non-hydrolysable chemical moiety with tryptamine precursors, which can be obtained synthetically, and which are analogue to tryptamines naturally occurring in psilocybin containing mushrooms, preferably in psilocybin containing mushrooms belonging to the Psilocybe genus.

Further, the invention relates to a composition comprising at least one of said non-hallucinogenic tryptamine compounds of formula I and/or formula II, optionally in combination with at least one monoamine oxidase inhibitor (MAOI) compound and/or with at least one antioxidant selected from carotenoids.

A further object of the invention are the compounds of formula I and/or formula II of claim 1 for use as a medicament.

Lastly, the invention relates to the compounds according to formula I and/or II and to the composition containing them, for use in the treatment of fibromyalgia, spinal cord injury-induced chronic neuropathic pain, neuropathic pain associated with diabetic peripheral neuropathy, post-herpetic neuralgia, chronic musculoskeletal pain, pain from chemotherapy associated neuropathy and/or a TNF-α-induced inflammatory disease.

According to a preferred embodiment, the TNF-α-induced inflammatory disease is selected from rheumatoid arthritis, juvenile idiopathic arthritis, psoriatic arthritis, ankylosing spondylitis, Crohn's disease, ulcerative colitis, plaque psoriasis, hidradenitis suppurativa, uveitis, pathological ocular neovascularization, reducing scarring in the eye, macular degeneration, neovascularization, retinochoroidal, corneal neovascularization, keratitis, and/or irritable bowel syndrome.

### BRIEF DESCRIPTION OF THE DRAWINGS

FIG. 1 is a graphical presentation of results from example 1.

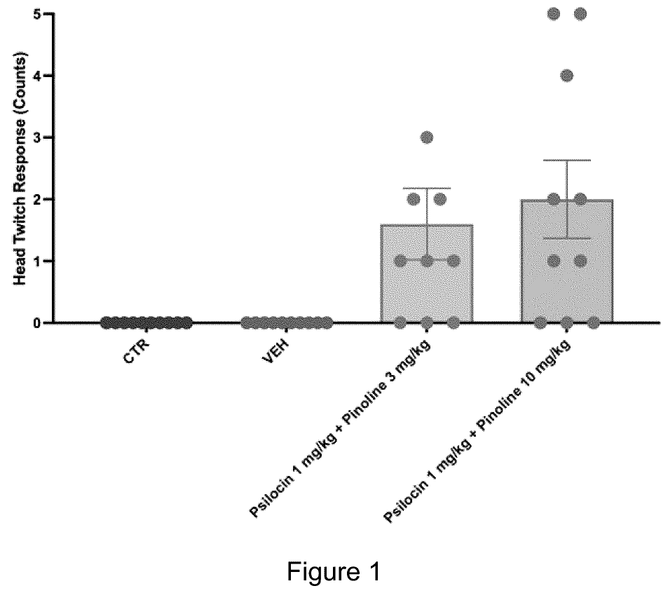

FIG. 2 is a graphical presentation of results from example 1.

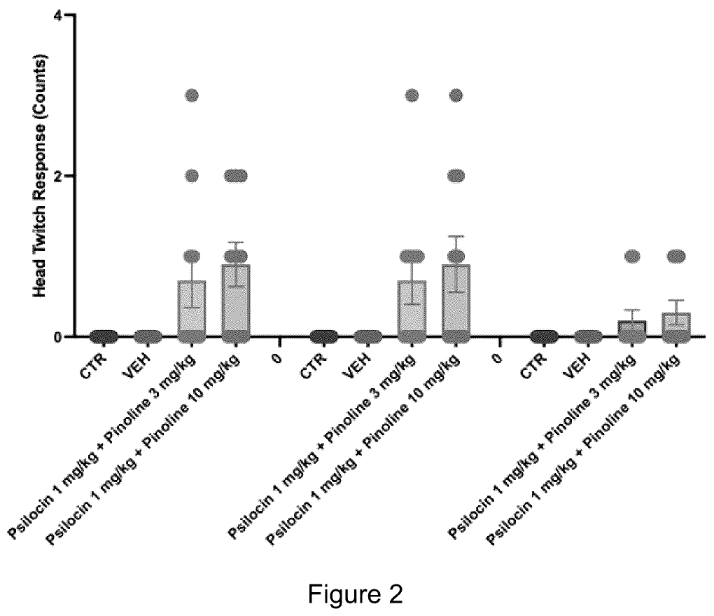

FIG. 3 is a graphical presentation of results from example 2.

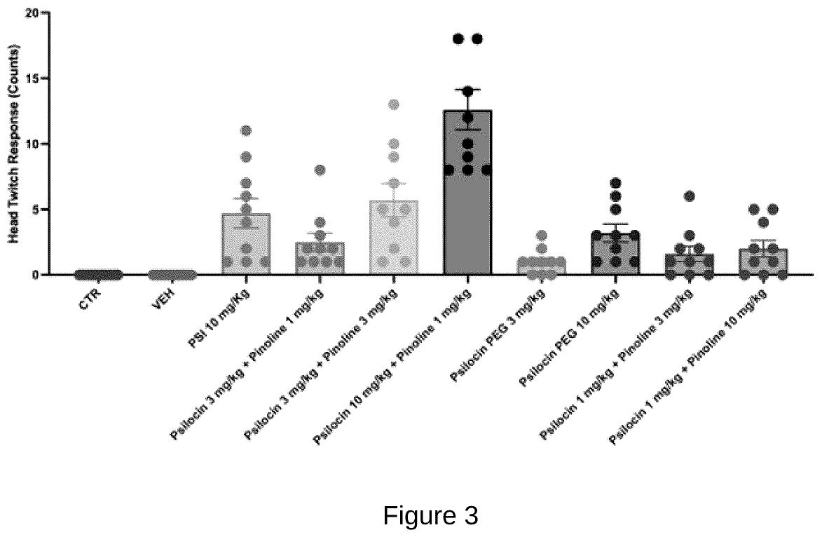

FIG. 4 is a graphical presentation of results from example 2.

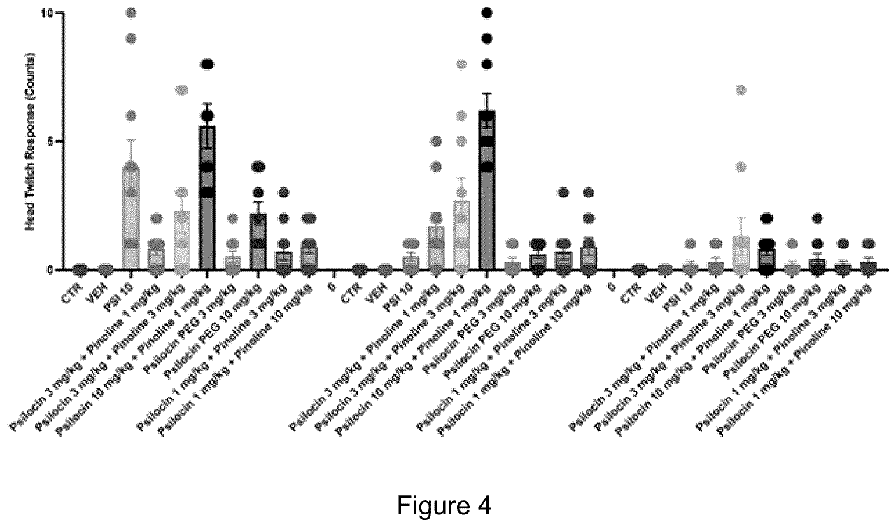

### DETAILED DESCRIPTION

The present invention relates to novel non-hallucinogenic tryptamine compounds of formula I and/or formula II:

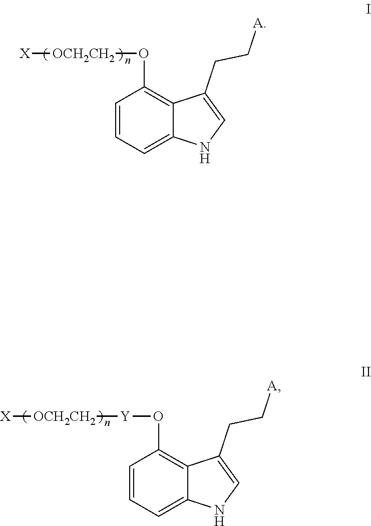

wherein:
A is —NR₁R₂ or —N⁺R₁R₂R₃;
R₁, R₂ and R₅ are each independently selected from the group consisting of H, CH₃ or an alkyl group, preferably selected from C₂H₅, C₃H₇ and (CH₃)₂CH, more preferably R₁, R₂ and R₅ are each CH₃;
the group (OCH₂CH₂)ₙ is oligo-ethylene glycol, wherein preferably n is selected from 1 and 50, more preferably n is selected from 3 and 12;
X is selected from the group consisting of H, CH₃, CH₂CH₂NH₂, CH₂CH₂SH, CH₂CH₂COOH, halogen or CH₂CH₂PO(OH)₂, with the proviso that X cannot be H in formula I;
Y is a C₁-C₃₀ alkyl spacer, optionally substituted with one or more hydroxy group, preferably a C₂-C₁₈ alkyl spacer, preferably a C₄-C₁₆ alkyl spacer, wherein the alkyl spacer optionally contains oxygen, nitrogen, sulfur, 1,2,3-triazole and/or a further oligo- or polyethylene glycol chain;
and/or pharmaceutically acceptable salts, derivatives, hydrate, or solvate and/or combinations thereof,
with the proviso that when R₁, R₂ and X are each CH₃ in formula I, n is not 4 or 6.

Particularly preferred are the compounds according to the above formula II, wherein Y is selected from

—(OCH₂CHOHCHOHCH₂O)—(CH₂)ₚ— or

—O—(CH₂)ₘ—(OCH₂CHOHCHOHCH₂O)—(CH₂)ₚ—,

and wherein m and p are each independently selected from 2 and 8, preferably between 4 and 6.

The compounds according to the above formula II, wherein n is selected from 3 and 12, preferably from 4 and 6, and more preferably wherein n is 6, and/or X is CH₃, are particularly preferred.

According to a further embodiment, the following compounds according to the above formula II are particularly preferred:

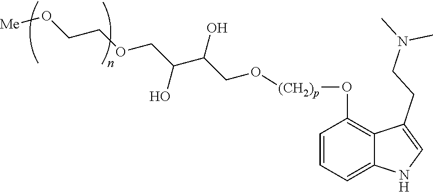

wherein n is 6, and p is selected from 4 and 6; and/or

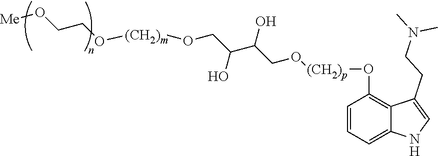

wherein n is 6, m and p are each independently selected from 4 and 6.

The compounds of formula I or formula II and/or pharmaceutically acceptable salts, derivatives, hydrate, or solvate and/or combinations thereof, with the proviso that when R₁, R₂ and X are each CH₃ in formula I, n is not 4 or 6 can be obtained using a process comprising a conjugation step of a non-hydrolysable chemical moiety with tryptamine precursors, which can be obtained synthetically, and which are analogue to tryptamines naturally occurring in psilocybin containing mushrooms, preferably in psilocybin containing mushrooms belonging to the Psilocybe genus. The non-hydrolysable chemical moiety is selected from:

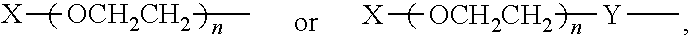

wherein:
the group (OCH₂CH₂)ₙ is oligo-ethylene glycol, wherein preferably n is selected from 1 and 50, more preferably n is selected from 3 and 12;
X is selected from the group consisting of H, CH₃, CH₂CH₂NH₂, CH₂CH₂SH, CH₂CH₂COOH, halogen or CH₂CH₂PO(OH)₂, with the proviso that X cannot be H in formula I; and
Y is a C₁-C₃₀ alkyl spacer, optionally substituted with one or more hydroxy group, preferably a C₂-C₁₈ alkyl spacer, preferably a C₄-C₁₆ alkyl spacer, wherein the alkyl spacer optionally contains oxygen, nitrogen, sulfur, 1,2,3-triazole and/or a further oligo- or polyethylene glycol chain.

According to a preferred embodiment, psilocybin containing mushrooms belonging to the Psilocybe genus comprise Psilocybe atlantis, Psilocybe azurenscens, Psilocybe bohemica, Psylocibe baeocystis, Psilocybe cyanescens, Psilocybe cubensis, Psilocybe tampanensis, Psilocybe hoogshagenii Psilocybe mexicana, Psilocybe ovoideocystidiata, Psilocybe semilanceata Psilocybe weraroa, Psilocybe stuntzii, Psilocybe cyanofibrillosa, Psilocybe zapotacorum, Psilocybe yungensis, Psilocybe liniformans, Psilocybe xalapensis, Psilocybe venenata, Psilocybe subtropicalis, Psilocybe singer, Psilocybe schultesii, Psilocybe rostrata, Psilocybe quebecensis, Psilocybe pintonii, Psilocybe puberula, Psilocybe mairei, Psilocybe laurae, Psilocybe kumaenorum, Psilocy beheimii, Psilocy begalindoi, Psilocybe fmetaria, Psilocy beegonii, Psilocybe dumontii, Psilocybe carbonaria, Psilocybe cordispora, Psilocybe bispora, Psilocybe aucklandii, and combinations thereof.

The above-described non-hydrolysable chemical moiety, namely oligo- or polyethylene glycol optionally functionalized and optionally containing a spacer, makes the tryptamine compounds of formula I and/or II unable to cross the blood brain barrier (BBB). Hence, the pharmacological activity of tryptamine compounds of formula I and/or II takes place only at a peripheral level, namely on peripheral 5-HT_{2A} receptors, thus avoiding secondary undesired effect on central nervous systems, such as hallucinations.

In view of the above, compounds of formula I and/or formula II of claim 1 can be used as a medicament, in particular they are non-hallucinogenic anti-inflammatory drugs, useful in the treatment of fibromyalgia, spinal cord injury-induced chronic neuropathic pain, neuropathic pain associated with diabetic peripheral neuropathy, post-herpetic neuralgia, chronic musculoskeletal pain, pain from chemotherapy associated neuropathy and/or a TNF-α-induced inflammatory disease.

According to a preferred embodiment, the TNF-α-induced inflammatory disease is selected from rheumatoid arthritis, juvenile idiopathic arthritis, psoriatic arthritis, ankylosing spondylitis, Crohn's disease, Crohn's disease, ulcerative colitis, plaque psoriasis, hidradenitis suppurativa, uveitis, pathological ocular neovascularization, reducing scarring in the eye, macular degeneration, neovascularization, retinochoroidal, corneal neovascularization, keratitis, and/or irritable bowel syndrome.

It is an object of the present invention also a composition comprising at least one non-hallucinogenic tryptamine compound of formula I and/or formula II above described.

According to a preferred embodiment, the composition of the invention further comprises at least one MAOI compound, to inhibit the metabolic breakdown and clearance of the non-hallucinogenic tryptamine compounds of formula I and/or formula II.

Monoamine oxidases (MAOs) are metabolic enzymes attached to cytosolic side of the outer membrane of mitochondria of neuronal, glial and several cell types. Specifically, they catalyze the oxidative deamination of neuroactive and vasoactive biogenic compounds (including serotonin and tryptamines) and xenobiotic amines into the corresponding aldehyde and ammonia, both in the central nervous system and peripheral tissues. Several monoamine oxidase inhibitors (MAOIs) have been extensive employed as antidepressants and neuroprotective agents in Parkinson's disease, as well as in the treatment of anxiety. There are two main isoforms, MAO-A and MAO-B. Monoamine oxidase A is predominantly responsible for the metabolism of psilocin (Reniers et al., “Synthesis and evaluation of β-carboline derivatives as potential monoamine oxidase inhibitors”, Bioorg. Med. Chem. (2011) v. 19 (1), p. 134-44).

According to a preferred embodiment, the at least one MAOI compound which can be optionally added to the composition of the invention is selected from the group consisting of norharmane, perlolyrine, harmol, cordysinins tetrahydroharmine, 6-methoxyharmalan, harmalan, harmaline, harmalol, dihydro-β-carbolines (DHβC), tetrahydro-β-carboline (THβC), methyl-tetrahydro-β-carboline MTHβC, pinoline, 1-trichloromethyl-1,2,3,4-tetrahydro-b-carboline (TaClo), 6-methoxytetrahydroharmalan, ethyl β-carboline-3-carboxylate (βCCE), β-carboline-3-carboxylate (βCCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, lucartamides D, plakortamines C, eudistomidins, threctandramine or fascaplysin and/or salts, derivatives, hydrate, or solvate and/or combinations thereof.

According to a further preferred embodiment, the at least one MAOI is selected from norharmane, perlolyrine, harmol, cordysinins tetrahydroharmine and/or salts, derivatives, hydrate, or solvate and/or combinations thereof.

Moreover, the composition of the invention can also comprise at least one antioxidant selected from carotenoids, preferably selected from carotenoids obtained by plants extraction and/or microbial fermentation.

According to a further preferred embodiment, the at least one antioxidant selected from carotenoids is α-carotene, β-carotene, lycopene, astaxantin and/or zeaxanthin.

The molar ratio between the at least one non-hallucinogenic tryptamine compound of formula I and/or formula II of the invention and the at least one MAOI compound, preferably selected from norharmane, perlolyrine, harmol, cordysinins tetrahydroharmine, 6-methoxyharmalan, harmalan, harmaline, harmalol, dihydro-β-carbolines (DHβC), tetrahydro-β-carboline (THβC), methyl-tetrahydro-β-carboline MTHβC, pinoline, 1-trichloromethyl-1,2,3,4-tetrahydro-b-carboline (TaClo), 6-methoxytetrahydroharmalan, ethyl β-carboline-3-carboxylate (βCCE), β-carboline-3-carboxylate (βCCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, lucartamides D, plakortamines C, eudistomidins, threctandramine, fascaplysin and/or salts, derivatives, hydrate, or solvate and/or combinations thereof is comprised between about 10:1 to about 1:10, preferably comprised between about 100:1 to about 1:100, more preferably comprised between about 1,000:1 to about 1:1,000 and further more preferably comprised between 10,000:1 to about 1:10,000.

According to a further preferred embodiment, the composition of the invention comprises also at least one antioxidant selected from carotenoids, preferably selected from carotenoids obtained by plants extraction and/or microbial fermentation. More preferably, the at least one antioxidant selected from carotenoids is α-carotene, β-carotene, lycopene, astaxantin and/or zeaxanthin.

The composition of the invention can be used in the treatment of fibromyalgia, spinal cord injury-induced chronic neuropathic pain, neuropathic pain associated with diabetic peripheral neuropathy, post-herpetic neuralgia, chronic musculoskeletal pain, pain from chemotherapy associated neuropathy and/or a TNF-α-induced inflammatory disease.

According to a preferred embodiment, the TNF-α-induced inflammatory disease is selected from rheumatoid arthritis, juvenile idiopathic arthritis, psoriatic arthritis, ankylosing spondylitis, Crohn's disease, ulcerative colitis, plaque psoriasis, hidradenitis suppurativa, uveitis, pathological ocular neovascularization, reducing scarring in the eye, macular degeneration, neovascularization, retinochoroidal, corneal neovascularization, keratitis, and/or irritable bowel syndrome.

### EXAMPLES

### Example 1—Edema Testing

A study was performed using male Sprague-Dawley rats (200-250 g; Envigo, Milan, Italy). Food and water were accessible ad libitum. Rats were concurrently subjected to a singular subplantar injection of CAR (100 μL of saline solution containing 1% CAR). Paw edema was measured with a plethysmometer (Ugo Basile, Comerio, Varese, Italy) [Salvemini, D.; Wang, Z.-Q.; Wyatt, P. S.; Bourdon, D. M.; Marino, M. H.; Manning, P. T.; Currie, M. G. Nitric oxide: A key mediator in the early and late phase of carrageenan-induced rat paw inflammation. Br. J. Pharmacol. 1996, 118, 829-838] prior to CAR injection and every hour for 6 h. Edema was expressed as the increase in paw volume (mL) after CAR injection relative to the pre-injection value for all animals. Scores were expressed as paw volume difference (mL).

Mechanical allodynia was evaluated using a dynamic plantar Von Frey hair aesthesiometer on time 0 and 1, 2, 3, 4, 5, and 6 days post-injection (Bio-EVF4; Bioseb, Vitrolles, France) as previously described (D'Amico, R.; Fusco, R.; Siracusa, R.; Impellizzeri, D.; Peritore, A. F.; Gugliandolo, E.; Interdonato, L.; Sforza, A. M.; Crupi, R.; Cuzzocrea, S.; et al. Inhibition of P2X7 Purinergic Receptor Ameliorates Fibromyalgia Syndrome by Suppressing NLRP3 Pathway. Int. J. Mol. Sci. 2021, 22, 6471. https://doi.org/10.3390/ijms22126471). The device encloses a force transducer furnished with a plastic tip. When pressure is applied to the tip, the force applied is recorded. The tip was applied to the plantar area of the hind leg, and a rising upward force was exerted until the paw was withdrawn. The withdrawal threshold was defined as the force, expressed in grams, at which the mouse removed the paw.

Injection of CAR into the hind paw of rats led to a significant time-dependent increase in paw volume. CAR-induced paw edema was significantly reduced by treatment with Psilocin+Pinoline. The administration used was: (a) Psilocin 1 mg/Kg+Pinoline 3 mg/Kg, and (b) Psilocin 1 mg/Kg+Pinoline 10 mg/Kg. Both reduced the paw volume. No difference was detected between Psilocin 1 mg/Kg+Pinoline 3 mg/Kg and Psilocin 1 mg/Kg+Pinoline 10 mg/Kg.

To investigate whether Psilocin+Pinoline were able to reduce pain associated with CAR injection, a Von Frey test was performed. No significant differences were observed in mechanical hyperalgesia in all animals at time 0. CAR injection reduced the paw-withdrawal threshold in response to von Frey hair stimulation at the other timepoints.

It was found that administration of Psilocin 1 mg/Kg+Pinoline 3 mg/Kg and Psilocin 1 mg/Kg+Pinoline 10 mg/Kg significantly increased the paw-withdrawal threshold as compared to CAR group. No difference was detected between Psilocin 1 mg/Kg+Pinoline 3 mg/Kg and Psilocin 1 mg/Kg+Pinoline 10 mg/Kg.

In conclusion, Psilocin 3 mg/Kg+Pinoline 1 mg/Kg and Psilocin 10 mg/Kg+Pinoline 1 mg/Kg showed the same anti-inflammatory and analgesic effects of Psilocin administration at 3 and 10 mg/Kg. Additionally, Psilocin 1 mg/Kg+Pinoline 3 mg/Kg and Psilocin 1 mg/Kg+Pinoline 10 mg/Kg showed anti-inflammatory and analgesic effects.

### Example 2—Head Twitch Testing

The study of example 2 was performed using C57BL/6J male mice (25-30 g; Envigo, Milan, Italy). The animals were divided into groups of 10 animals each: (a) control, (b) vehicle, and (c) combinations of psilocyn (1 mg/kg) and pinoline (3 or 10 mg/kg). The treated mice were left to fast in the 12 hours prior to administration. Psilocyn and pinoline were resuspended in a solution of EtOH 20%. Mice were subjected to a singular oral administration of compounds. The vehicle group was given the EtOH 20% solution.

After administration, the animals were placed individually in transparent plexiglass cages. They were observed for the next 30 minutes with the number of head twitch responses recorded for each. (See Halberstadt, Adam L., et al. “Differential contributions of serotonin receptors to the behavioral effects of indoleamine hallucinogens in mice.” Journal of psychopharmacology 25.11 (2011): 1548-1561.) (Sun, Yina, et al. “Effects of low-doses of methamphetamine on d-fenfluramine-induced head-twitch response (HTR) in mice during ageing and c-fos expression in the prefrontal cortex.” BMC neuroscience 24.1 (2023): 1-17.).

During the overall 30-minute observation period there was a significant increase in the number of head shaking in both groups administered with the combination of psilocyn and pinoline. The control and vehicle-only subjects had no measurable head twitching.

When divided into 10-minute time slots, the administrations of Psilocyn and Pinoline led to an increase in the number of head shakes in the 10 and 20 minute slots with a slight decrease in the last 10 minute slot.

The head twitch test results of this example support the hypothesis that pinoline administration increased the psilocin activity. As an MAO inhibitor, pinoline prolonged the psilocin (1 mg/kg) activity at 3 mg/kg and 10 mg/kg.

After 10 minutes from the treatments, psilocin at 10 mg/kg and psilocin PEG 10 mg/kg showed an important number of head movements.

After 11-20 minutes from administration, the effect of psilocin at 10 mg/kg and psilocin PEG 10 mg/kg were finished while the administration of (psilocin 1 mg/kg+pinoline 3 mg/kg), (psilocin 1 mg/kg+pinoline 10 mg/kg), (psilocin 3 mg/kg+pinoline 3 mg/kg) and (psilocin 10 mg/kg+pinoline 1 mg/kg) still displayed an effect in this test.

After 21-30 minutes from administration, the effects of (psilocin 3 mg/kg+pinoline 3 mg/kg) and (psilocin 10 mg/kg+pinoline 1 mg/kg) were still higher than the (psilocin at 10 mg/kg and Psilocin PEG 10 mg/kg).

1. A compound of the formula I or II: 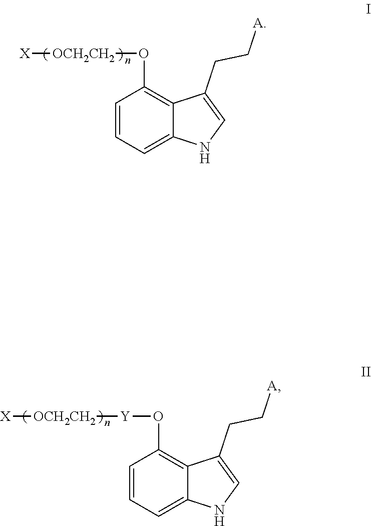
wherein:
A is —NR₁R₂ or —N⁺R₁R₂R₃;
R₁, R₂ and R₅ are each independently selected from the group consisting of H, CH₃ or an alkyl group, preferably selected from C₂H₅, C₃H₇ and (CH₃)₂CH, more preferably R₁, R₂ and R₅ are each CH₃;
the group (OCH₂CH₂)ₙ is oligo-ethylene glycol, wherein preferably n is selected from 1 and 50, more preferably n is selected from 3 and 12;
X is selected from the group consisting of H, CH₃, CH₂CH₂NH₂, CH₂CH₂SH, CH₂CH₂COOH, halogen or CH₂CH₂PO(OH)₂, with the proviso that X cannot be H in formula I;
Y is a C₁-C₃₀ alkyl spacer, optionally substituted with one or more hydroxy group, preferably a C₂-C₁₈ alkyl spacer, preferably a C₄-C₁₆ alkyl spacer, wherein the alkyl spacer optionally contains oxygen, nitrogen, sulfur, 1,2,3-triazole and/or a further oligo- or polyethylene glycol chain;
and/or pharmaceutically acceptable salts, derivatives, hydrate, or solvate and/or combinations thereof,
with the proviso that when R₁, R₂ and X are each CH₃ in formula I, n is not 4 or 6.

2. A compound according to claim 1, wherein Y of formula II is selected from
—(OCH₂CHOHCHOHCH₂O)—(CH₂)ₚ— or
—O—(CH₂)ₘ—(OCH₂CHOHCHOHCH₂O)—(CH₂)ₚ—,
and wherein m and p are each independently selected from 2 and 8, preferably between 4 and 6.

3. A compound according to claim 2, wherein n is selected from 3 and 12, preferably from 4 and 6, and more preferably n is 6, and/or X is CH₃.

4. A compound according to claim 2, having the following formula: 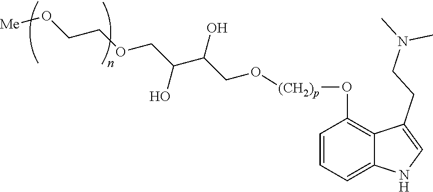
wherein n is 6, and p is selected from 4 and 6.

5. A compound according to claim 2, having the following formula: 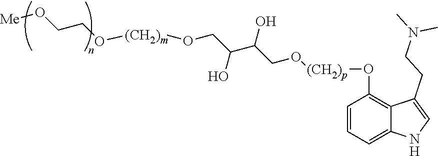
wherein n is 6, m and p are each independently selected from 4 and 6.

6. A composition having at least one non-hallucinogenic tryptamine compound of formula I and/or formula II.

7. A composition according to claim 6, further comprising at least one MAOI compound, preferably selected from the group consisting of norharmane, perlolyrine, harmol, cordysinins tetrahydroharmine, 6-methoxyharmalan, harmalan, harmaline, harmalol, dihydro-β-carbolines (DHβC), tetrahydro-β-carboline (THβC), methyl-tetrahydro-β-carboline MTHβC, pinoline, 1-trichloromethyl-1,2,3,4-tetrahydro-b-carboline (TaClo), 6-methoxytetrahydroharmalan, ethyl β-carboline-3-carboxylate (βCCE), β-carboline-3-carboxylate (βCCM), manzamine A, manzamine X, 6-deoxymanzamine X, manzamine Y, 8-hydroxymanzamine A, 8-methoxymanzamine A, 6-hydroxymanzamine A, 3,4-dihydromanzamine A, ent-8-hydroxymanzamine A, ent-manzamine F, neo-kauluamine, xestomanzamine B, hyrtioerectines A, gesashidine A, plakortamines A, plakortamines B, lucartamides D, plakortamines C, eudistomidins, threctandramine or fascaplysin and/or salts, derivatives, hydrate, or solvate and/or combinations thereof.

8. A composition according to claim 7, wherein the at least one MAOI is selected from norharmane, perlolyrine, harmol, cordysinins tetrahydroharmine and/or salts, derivatives, hydrate, or solvate and/or combinations thereof.

9. A composition according to claim 7, wherein a molar ratio between the at least one non-hallucinogenic tryptamine compound of formula I and/or formula II and the at least one MAOI compound, is between about 10,000:1 to about 1:10,000.

10. A composition according to claim 6, further comprising at least one antioxidant selected from carotenoids, preferably selected from carotenoids obtained by plants extraction and/or microbial fermentation.

11. A composition according to claim 10, wherein the at least one antioxidant zeaxanthin.

12. A process for the treatment of a patient suffering from fibromyalgia, spinal cord injury-induced chronic neuropathic pain, neuropathic pain associated with diabetic peripheral neuropathy, post-herpetic neuralgia, chronic musculoskeletal pain, pain from chemotherapy associated neuropathy and/or a TNF-α-induced inflammatory disease by steps that comprise administering to said patient a therapeutically effective amount of a compound according to formula I and/or formula II.

13. A process according to claim 12, wherein the TNF-α-induced inflammatory disease is selected from rheumatoid arthritis, juvenile idiopathic arthritis, psoriatic arthritis, ankylosing spondylitis, Crohn's disease, ulcerative colitis, plaque psoriasis, hidradenitis suppurativa, uveitis, pathological ocular neovascularization, reducing scarring in the eye, macular degeneration, neovascularization, retinochoroidal, corneal neovascularization, keratitis, and/or irritable bowel syndrome.
